(19) United States
(12) Patent Application Publication
(10) Pub. No.: US 2025/0143895 A1
(43) Pub. Date: May 8, 2025

(54) PROSTHETIC FOOT WITH ENHANCED STABILITY AND ELASTIC ENERGY RETURN

(71) Applicant: Össur Iceland ehf, Reykjavík (IS)

(72) Inventors: Arinbjorn Viggo Clausen, Reykjavik (IS); Bjarni Andresson, Seltjarnarnes (IS); Vilhjalmur Freyr Jonsson, Reykjavik (IS); Christophe Lecomte, Reykjavik (IS)

(21) Appl. No.: 19/014,796

(22) Filed: Jan. 9, 2025

Related U.S. Application Data
(63) Continuation of application No. 17/677,194, filed on Feb. 22, 2022, now Pat. No. 12,220,330.
Application No. 17/677,194 is a continuation of application No. 16/374,055, filed on Apr. 3, 2019, now Pat. No. 11,285,024.
Application No. 16/374,055 is a continuation of application No. 15/386,366, filed on Dec. 21, 2016, now Pat. No. 10,369,019.
Application No. 15/386,366 is a continuation of application No. 14/188,216, filed on Feb. 24, 2014, now Pat. No. 9,561,118.
(60) Provisional application No. 61/769,405, filed on Feb. 26, 2013.
(60) Provisional application No. 61/770,212, filed on Feb. 27, 2013.

(51) Int. Cl. A61F 2/66; A61F 2/50; A61F 2/70; A61F 2/74; A61F 2/76

(52) U.S. Cl. CPC A61F 2/6607; A61F 2/66; A61F 2/70; A61F 2002/5006; A61F 2002/503; A61F 2002/5033; A61F 2002/5079; A61F 2002/6657; A61F 2002/6664; A61F 2002/6671; A61F 2002/701; A61F 2/74; A61F 2002/7615

(57) ABSTRACT

A prosthetic foot comprises an attachment member and two or more flexible members. The attachment member can include a connector configured to connect the attachment member to a user or another prosthetic device. The two or more flexible members can be rotatably attached to the attachment member by rotatable joints such that the flexible members can both rotate and flex relative to the attachment member when the prosthetic foot contacts the ground.

### CROSS-REFERENCE TO RELATED APPLICATIONS

This application is a continuation of U.S. application Ser. No. 17/677,194, filed Feb. 22, 2022, which is a continuation of U.S. application Ser. No. 16/374,055, filed Apr. 3, 2019, now U.S. Pat. No. 11,285,024, issued Mar. 29, 2022, which is a continuation of U.S. application Ser. No. 15/386,366, filed Dec. 21, 2016, now U.S. Pat. No. 10,369,019, issued Aug. 6, 2019, which is a continuation of U.S. application Ser. No. 14/188,216, filed Feb. 24, 2014, now U.S. Pat. No. 9,561,118, issued Feb. 7, 2017, which claims priority benefit of U.S. Provisional Application Nos. 61/769,405, filed Feb. 26, 2013 and 61/770,212, filed Feb. 27, 2013, the entirety of each of which are hereby incorporated by reference herein and should be considered a part of this specification.

### BACKGROUND

### Field

The present application relates to prosthetic feet and other prosthetic devices including a spring, and more particularly to prosthetic feet and other prosthetic devices having one or more flexible members between two or more joints (e.g., pivots) and allowing for variable stiffness during use.

### Description of the Related Art

In the field of prosthetics, particularly prosthetic feet, it is desirable to provide a high level of functionality with reliable performance. Further, as each user is different, it is desirable to provide a prosthesis that can be adapted to the particular needs of each individual user.

### SUMMARY

Particularly in the area of prosthetic feet, it is desirable to provide a prosthesis that provides stability throughout the gait cycle and in other activities such as stance. Further, during movement it is often desirable for a prosthetic foot to absorb and return elastic energy, while having enhanced energy conservation during ambulation. Even further, it is desirable for a prosthetic foot to be adjustable to an individual who may have various weights, heights, stride lengths, etc., as well as for prosthetic foot designs to allow for a variable stiffness, depending on the activity level of the amputee.

In accordance with one embodiment, a prosthetic foot is provided having one or more flexible members between two or more joints (e.g., pivots) to provide improved control and stability during a stance phase of gait cycle (e.g., provide more movement during stance). In one embodiment, the prosthetic foot is purely a mechanical foot. In another embodiment the prosthetic foot can include an actuator. In some embodiments, the actuator can be an active actuator (e.g., an electric motor) that can be selectively actuated (e.g., via an electric controller) to impart mechanical motion to the prosthetic foot (e.g., to change the orientation of the prosthetic ankle during a swing phase of gait cycle to dorsiflexion and then to plantarflexion). In another embodiment, the actuator can be a passive actuator (e.g., resilient member, spring or stiff beam).

In another embodiment, a prosthetic foot is provided with a variable stiffness control, which allows the stiffness of the prosthetic foot to be adjusted for different types of gait. In some embodiments, the variable stiffness control is mechanically actuatable (e.g., actuated manually by a user) to vary the stiffness of one or more elastic elements of the prosthetic foot (e.g., by changing the length of a lever arm of an elastic element, or by varying a gap between adjacent elastic elements). In another embodiment, the variable stiffness control can be automatically or actively adjusted during ambulation by the user (e.g., automatic adjustment of a lever arm of an elastic element, or active varying of a gap between adjacent elastic elements), e.g., based on the activity level of the user or the phase of gait cycle. In some embodiments the variable stiffness control can be automatically adjusted based on a sensed parameter of gait (e.g., sensed with one or more sensors on the prosthetic device).

In still another embodiment, the prosthetic foot or device can include a housing or adapter (e.g., for coupling the prosthetic foot or device to another prosthetic component) with a mechanism that provides for flexible motion in one or more planes (e.g., sagittal, coronal, transverse) so as to allow motion of the housing or adapter in a medial-lateral, anterior-posterior, or torsional direction. In one embodiment, where the prosthetic device is a prosthetic foot, the housing or adapter can be located generally at a location associated with a natural human ankle, and provide for motion similar to that of a natural human ankle. In some embodiments, the mechanism can include one or more slots or openings in one or more surfaces of the housing or adapter (e.g., slots on medial and lateral surfaces of the housing or adapter), that movably receive one or more pins, axles or joint members that connect the housing or adapter with other components (e.g., clastic elements or foot plates) of the prosthetic foot.

In one embodiment, a prosthetic foot comprises an attachment member and two or more flexible members. The attachment member can include a connector configured to connect the attachment member to a user or another prosthetic device. The two or more flexible members can be rotatably attached to the attachment member by rotatable joints such that the flexible members can both rotate and flex relative to the attachment member when the prosthetic foot contacts the ground.

In another embodiment, a prosthetic foot can include an attachment member, two or more flexible members, and an adjustable fastening member. The attachment member can include a connector configured to connect the attachment member to a user or another prosthetic device. The two or more flexible members can attach to the attachment member. Further, the two or more flexible members can extend from the attachment member to a foot portion of the prosthetic foot and be substantially movable relative to each other along their lengths. The adjustable fastening member can be configured to fasten the two or more flexible members along the foot portion of the prosthetic foot. Further, fastening can be provided at a plurality of positions along the length of the two or more flexible members to change the flexibility and resistance of the two or more flexible members.

In further embodiments, a prosthetic foot can include an attachment member, two or more flexible members, and a variable stiffness control member. The attachment member can include a connector configured to connect the attachment member to a user or another prosthetic device. The two or more flexible members can attach to the attachment member and can extend from the attachment member to a foot portion of the prosthetic foot. The flexible members can be substantially movable relative to each other along their lengths. However, the variable stiffness control member can be configured to adjust a length of a lever arm of the two or more flexible members along the foot portion of the prosthetic foot. For example, the variable stiffness control member can limit the relative motion between the flexible members.

In further embodiments, a prosthetic foot can include one or more flexible foot plates, an attachment member, and a means for modifying the stiffness of the prosthetic foot. The one or more flexible foot plates can be configured to bend along their lengths. The attachment member can include a connector configured to connect the attachment member to a user or another prosthetic device. The means for modifying the stiffness of the prosthetic foot can change the bending length of one or more of the flexible foot plates either prior to or during use.

In further embodiments, a prosthetic foot can include one or more elastic elements and an attachment member. The one or more elastic elements can be configured to bend along their lengths. The attachment member can include a connector configured to connect the attachment member to a user or another prosthetic device. Further, the attachment adapter can be connected to the one or more elastic elements via at least two pivotable joints. At least one of the elastic elements can extend between the at least two pivotable joints.

### BRIEF DESCRIPTION OF THE DRAWINGS

These and other features, aspects, and advantages of the invention disclosed herein are described below with reference to the drawings of preferred embodiments, which are intended to illustrate and not to limit the invention. Additionally, from figure to figure, the same reference numerals have been used to designate the same components of an illustrated embodiment. The following is a brief description of each of the drawings.

FIG. 1 is a perspective view of an embodiment of a prosthetic foot.

FIG. 2 is a front view of the prosthetic foot of FIG. 1.

FIG. 3 is a rear view of the prosthetic foot of FIG. 1.

FIG. 4 is a side view of the prosthetic foot of FIG. 1.

FIG. 4A is a side view of another embodiment of a prosthetic foot, similar to the prosthetic foot of FIG. 1.

FIG. 5 is a top view of the prosthetic foot of FIG. 1.

FIG. 6 is a bottom view of the prosthetic foot of FIG. 1.

FIG. 7 is a cross-sectional side view of the prosthetic foot of FIG. 1.

FIG. 8 is a view of the prosthetic foot of FIG. 1 with additional fasteners.

FIG. 9 is a perspective view of another embodiment of a prosthetic foot.

FIG. 10 is a front view of the prosthetic foot of FIG. 9.

FIG. 11 is a rear view of the prosthetic foot of FIG. 9.

FIG. 12 is a side view of the prosthetic foot of FIG. 9.

FIG. 13 is a top view of the prosthetic foot of FIG. 9.

FIG. 14 is a bottom view of the prosthetic foot of FIG. 9.

FIG. 15 is a cross-sectional side view of the prosthetic foot of FIG. 9.

FIG. 15A is a cross-sectional view of another embodiment of a prosthetic foot, similar to the prosthetic foot of FIG. 9.

FIG. 16 is a perspective view of another embodiment of a prosthetic foot.

FIG. 17 is a front view of the prosthetic foot of FIG. 16.

FIG. 18 is a rear view of the prosthetic foot of FIG. 16.

FIG. 19 is a side view of the prosthetic foot of FIG. 16.

FIG. 20 is a top view of the prosthetic foot of FIG. 16.

FIG. 21 is a bottom view of the prosthetic foot of FIG. 16.

FIG. 22 is a cross-sectional side view of the prosthetic foot of FIG. 16.

FIG. 23 is a view of the prosthetic foot of FIG. 16 with an additional fastener.

### DETAILED DESCRIPTION

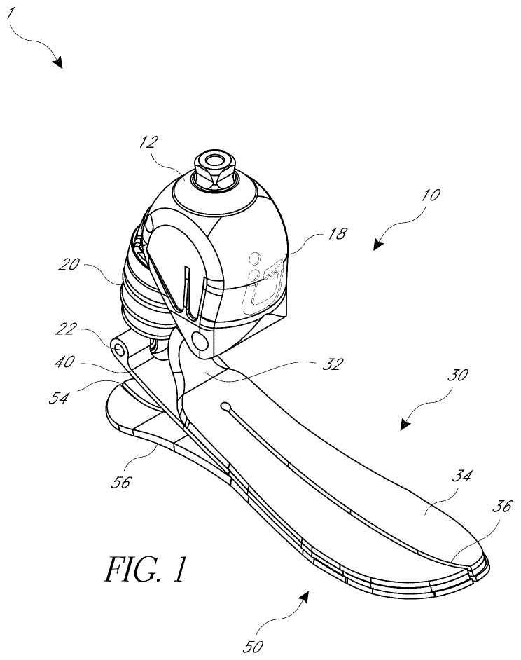

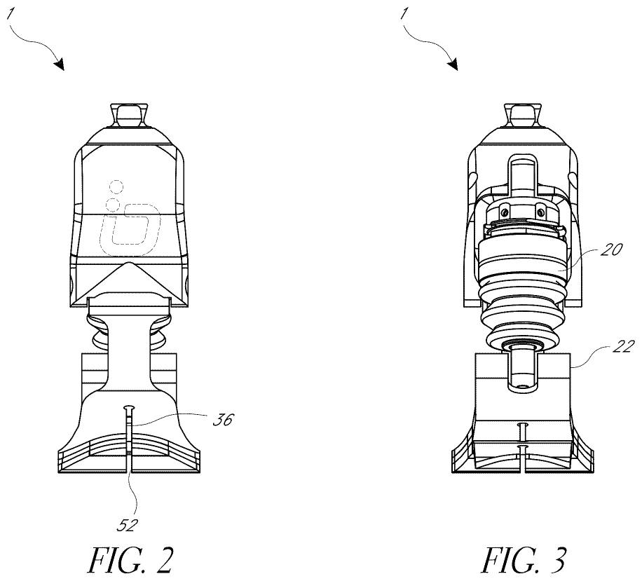

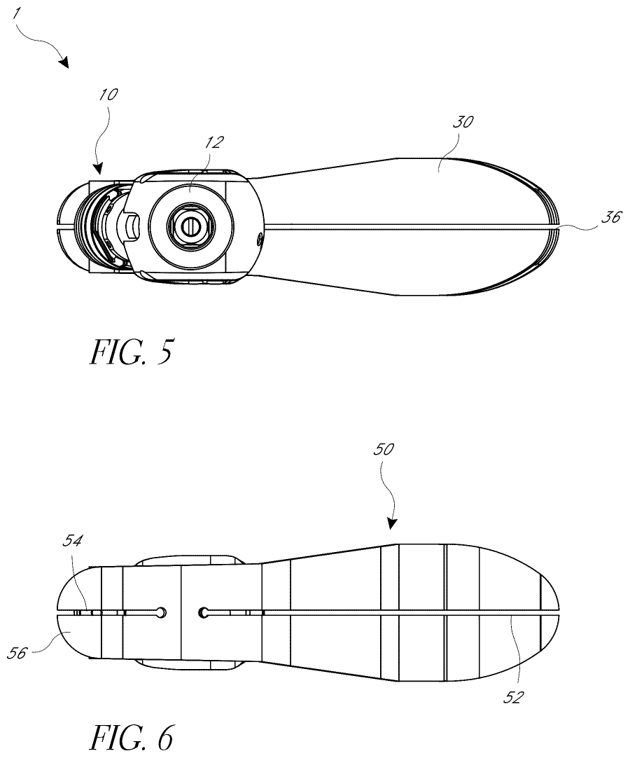

FIGS. 1-8 depict an embodiment of a prosthetic foot 1. The prosthetic foot 1 can attach to a user or to another prosthetic device with an attachment member 10. The attachment member 10 is depicted as including a first connection portion 12 shown as a pyramid connector. The pyramid connector can attach to a stump on a user, to another prosthetic device, or to any other appropriate object. Further, it will be understood that the first connection portion 12 can include attachment features other than a pyramid connector, such as a threaded hole or screw, a latch, a magnetic member, tube clamp, or other features.

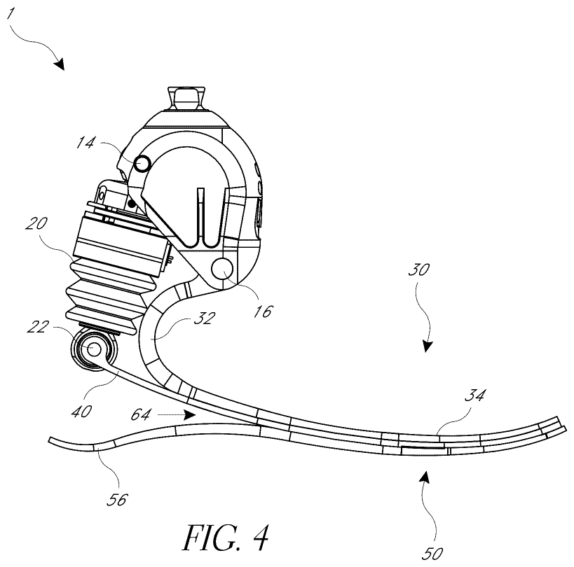

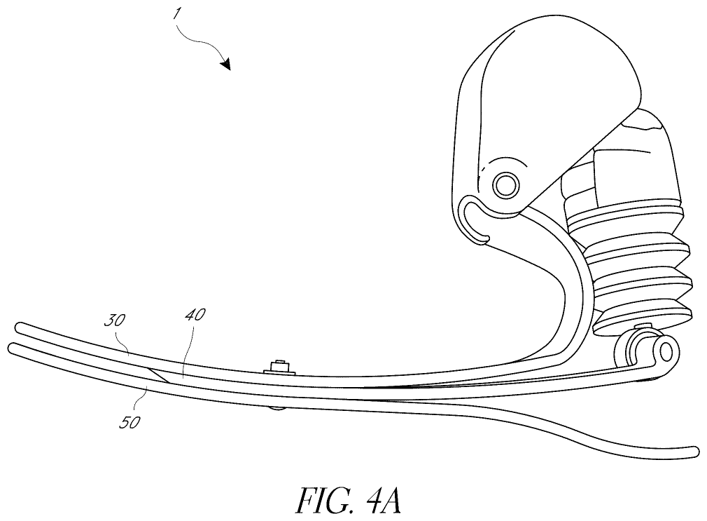

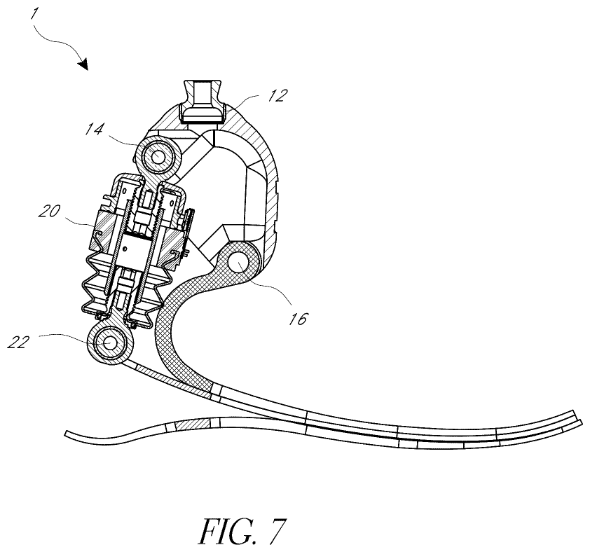

The attachment member 10 can additionally include second and third connection portions 14, 16 (see FIGS. 4, 7). The attachment member 10 can serve to provide a rigid connection between the connection portions 12, 14, 16. For example, the attachment member 10 can comprise a substantially rigid material such as aluminum, steel, titanium, other metals or metallic alloys, carbon fiber, composites, or substantially rigid plastics. However, in other embodiments the attachment member 10 can be configured to provide flexibility, potentially in multiple planes. Thus, in some embodiments the attachment member 10 can comprise a more flexible material or include flexible joints between separate components of the attachment member 10. For example, in some embodiments the attachment member 10 can have a flexible connection with the first connection portion 12, allowing for motion in the medial/lateral and/or anterior/posterior directions. Further, the connection may allow torsional flexibility with the first connection portion 12. In other embodiments, as further described below, the attachment member 10 can have a flexible connection with one or both of the second and third connection portions 14, 16.

Further, in some embodiments the attachment member 10 can include other features of a prosthetic foot such as sensors configured to measure, for example, the position and movement of the prosthetic foot, the position and movement of various joints and components on the prosthetic foot (such as the rotational position and movement at the connection portions 14, 16 and an actuator 20, as further discussed below), pressures and forces on various components of the prosthetic foot 1 (such as on the attachment member 10, the actuator 20, or the elastic members 30, 40, 50, further discussed below), and other measurable characteristics of the prosthetic foot. The sensors can additionally be configured to measure the prosthetic foot's environment, such as a terrain on which the prosthetic foot 1 moves. It will be understood that these sensors can be positioned on other elements of the prosthetic foot 1, such as the actuator 20, the elastic members 30, 40, 50, and other elements, further described below.

The attachment member 10 can also include electronics (e.g., computer processor). For example, the attachment member 10 can include electronics configured to receive information from the sensors, discussed above. Further, in some embodiments, the attachment member 10 can include electronics configured to communicate information (e.g., information from the sensors) to other electronic devices, such as to other prosthetic devices or to an external computer (e.g., via wired or wireless communication, such as RF communication). Such electronics may also be configured to receive information from other prosthetic devices or an external computer, such information potentially including information from other sensors and/or operational commands for the prosthetic foot 1.

The attachment member 10 can additionally include or define a cover 18. The cover 18 can protect various components of the prosthetic foot 1 such as electronics (as described above), the actuator 20 (describe below), or other components. In some embodiments the cover 18 can include open portions in the coronal plane, allowing flexibility of motion in the medial-lateral directions. In further embodiments the cover 18 can include open portions in the sagittal plane, allowing flexibility of motion in the anterior-posterior directions. In some embodiments, the open portions can be vertical or horizontal slots formed in the cover 18, to allow movement of pivot axles associated with any one of the connection portions 12, 14, 16.

As shown in FIG. 1, the attachment member 10 can connect to a first clastic member 30 at the third connection portion 16. In some embodiments the third connection portion 16 can provide a rotatable connection, although non-rotatable connections can also be used. In some embodiments, the rotation can be provided by an axle firmly mounted to the attachment member 10, about which the first clastic member 30 can rotate. In other embodiments, the first elastic member 30 can be fixed to the axle, and relative rotation can be allowed between the axle and the attachment member 30. In one embodiment, the first clastic member 30 can include or define a bushing or opening through which the axle extends. The first elastic member 30 can be formed from a sufficiently flexible material such as carbon fiber, though other suitable materials or combination of materials can be used (e.g., carbon and glass fibers). In other embodiments, the first elastic member 30 can be substantially inelastic, so as to provide a rigid connection. It will be understood that the other elastic members 40, 50 (described further below) can be formed of similar materials and have similar connections as the first elastic member 30.

Further, the first elastic member 30 can be formed into a shape configured to provide a desired flexibility or rigidity. As shown in FIG. 1, the elastic member 30 includes a C-shaped portion 32 at an upper portion (proximal portion) of the first clastic member, near the third connection portion 16. The C-shaped portion 32 is depicted as including an opening facing forward (e.g., the C-shaped portion 32 curves forwardly so that it is concave toward the front of the prosthetic foot), although in other embodiments the C-shaped portion can have an opening facing backward (e.g., the C-shaped portion can curve rearwardly so that it is concave toward the rear of the prosthetic foot). In some embodiments, the C-shaped portion 32 can bend more than 90 degrees, more than 110 degrees, 130 degrees, 150 degrees, or 170 degrees when unloaded. The bend of the C-shaped portion 32 can affect the resistance or flexibility of the first elastic member 30. Notably, this resistance or flexibility can be adjusted, as described further below.

In the embodiment of FIG. 1, the elastic member 30 can extend from the lower portion of the C-shaped portion 32 into a foot portion 34. The foot portion 34 of the clastic member 30 can be substantially flat and extend from a rear portion of the prosthetic foot 1 toward a toe region of the prosthetic foot 1. The foot portion 34 can further include a slit 36. As shown, the slit 36 extends longitudinally to a toe end of the clastic member 30 to separate the foot portion 34 into two or more foot members that can flex independently, although in some embodiments the slit 36 can be closed at the toe end (e.g., where at least one of the elastic members 30, 40, 50 are solid at a toe portion such that the slit terminates prior to the end of the at least one elastic member). As will be discussed further below, the slit 34 can allow the flexibility and resistance of the elastic member 30 to be altered. In another embodiment, the clastic members 30, 40, 50 can be monolithic without any slits.

As further shown in FIG. 4, the attachment member 10 can connect to an actuator 20 at the second connection portion 14. Like the third connection portion 16, the second connection portion 14 can be rotatable or non-rotatable. Notably, in FIG. 4 the third connection portion 16 is in a front portion of the attachment member 10, and the second connection portion 14 is in a rear portion of the attachment member 10. Similarly, the actuator 20 is located at a rear portion of the prosthetic foot 1. However, in other embodiments the actuator 20 can be positioned in a front portion of the prosthetic foot 1, as further described below.

The actuator 20 can be in a variety of forms and can be operated in a variety of ways, as described by way of example in U.S. patent application Ser. No. 11/367,049, issued Mar. 1, 2011 as U.S. Pat. No. 7,896,927, and U.S. patent application Ser. No. 12/816,968, published as U.S. 2010/0324698 on Dec. 23, 2010, both of which are incorporated herein by reference and should be considered a part of this specification. For example, the actuator 20 can be a powered actuator such as a screw motor, or a passive member such as an elastic member (e.g., a spring) or a chamber with a magnetorheologic fluid, or can be a hydraulic or pneumatic system. Further, the actuator 20 can be configured to operate in a variety of ways, as also discussed in U.S. Pat. No. 7,896,927 and US 2010/0324698. For example, the actuator 20 can be configured to extend or contract to assist a user during a gait cycle. For example, the actuator 20 can change the orientation of the prosthetic foot 1 to dorsiflexion and then to plantarflexion during a swing phase of gait cycle so that the toe portion of the prosthetic foot 1 is raised during the initial portion of swing phase. In another embodiment, the actuator 20 can change the orientation of the prosthetic foot 1 to plantarflexion when the user is in a relaxed (e.g., sitting) position. Further, such motion of the actuator 20 can change the flexibility or resistance of the elastic members 30, 40, 50, as further described below. In some embodiments, the actuator 20 can also enter a low power mode (e.g., hibernation mode), such as a relaxed mode or an inactive mode. For example, the actuator 20 may enter a low power mode during stance, as the embodiments described herein can provide greater stability during stance, as further described below. Advantageously, the low power mode allows for the conservation of battery power used to power the actuator 20, allowing the actuator 20 to be operated for longer periods of time between battery charging.

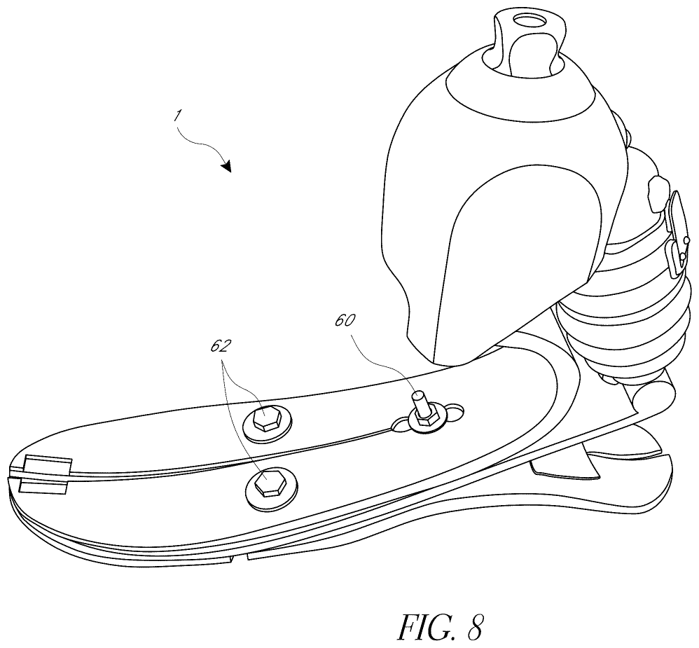

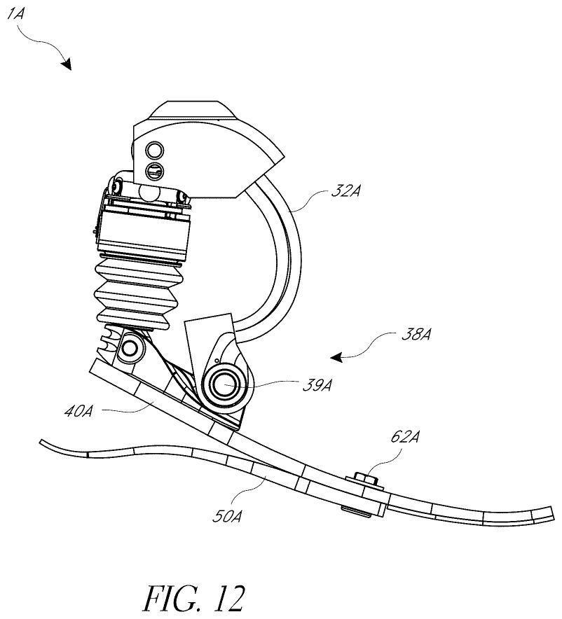

The actuator 20 is depicted as connecting to a second elastic member 40 at a fourth connection portion 22. Like the second and third connection portions 14, 16, the fourth connection portion 22 can be rotatable or non-rotatable. In one embodiment, the second elastic member 40 can include or define a bushing or opening through which an axle extends to provide a rotatable connection or pivot axis between the second elastic member 40 and the actuator 20. The second elastic member 40 can extend into a foot portion in a manner similar to the foot portion 34 of the first elastic member 30. In one embodiment, the second elastic member 40 can extend to a distal end of the prosthetic foot 1, so that the first and second elastic members 30, 40 extend to generally the same location at the distal end of the prosthetic foot 1. Further, the second elastic member 40 can include a slit similar to the slit 36 of the first elastic member 30. Even further, the second elastic member 40 can be composed of materials similar to those for the first elastic member, such as carbon fiber. As shown, the second elastic member 40 is disposed below the first elastic member 30, and extends tangentially forward and toward the first clastic member to abut the first elastic member 30 along the foot portion 34 of the first clastic member 30. Although the first and second elastic members 30, 40 are depicted as ending at approximately the same point at a toe portion of the prosthetic foot, in some embodiments the first elastic member 30 may extend further, or the second elastic member 40 may extend further. For example, as depicted in FIG. 4A, the first and third elastic members 30, 50 can extend further than the second elastic member 40, creating a gap between the first and third elastic members 30, 50. In other embodiments, a gap can be provided between the first and second elastic members 30, 40 in a toe region of the prosthetic foot, as shown in FIG. 8. As a further example, as depicted in, for example, FIG. 12 the third elastic member 50 (e.g., member 50A, described below) can end before a toe portion of the prosthetic foot 1, such as at a metatarsal region of the foot. The first and/or second elastic members 30, 40 (e.g., only second elastic member 40A in FIG. 12) can then extend past the third elastic member 50 to the toe portion.

The prosthetic foot 1 can further include a third elastic member 50. As shown, the third elastic member 50 can extend from a heel portion 56 (e.g., a cantilevered or free end) at a bottom and rear portion of the prosthetic foot 1. This heel portion 56, as shown, can be spaced from the actuator 20 and the second clastic member 40, curving downward toward and away from the actuator 20. From the heel portion 56, the third clastic member 50 can extend to a toe portion of the prosthetic foot 1, and can generally abut the foot portion second elastic member 40, as that member abuts the first elastic member 30. Further, the third elastic member 50 can have a slit along this foot portion that generally matches the slits in the first and second elastic members 30, 40. Additionally, as shown, the third elastic member 50 can include a heel slit 54 in the heel portion 56 of the elastic member.

As shown in the figures, the slit 36 in the first elastic member 30 can align with the slit in the second elastic member 40 and the slit 52 in the third elastic member 50 in the foot portion 34. In one embodiment, the prosthetic foot 1 can have a stiffness control member 60 that can be actuated to vary the stiffness of the prosthetic foot. In some embodiments, the stiffness control member 60 can be a fastening member 60 (e.g., bolt and nut, clamp, staple, rivet, etc.) that couples two or more of the elastic members 30, 40, 50 to each other, where the fastening member 60 can travel along the slit 36 or a slot defined at least partially by the slit, best shown in FIG. 8, or travel along a slot in the elastic members 30, 40, 50 where the elastic members do not have a slit. Attachment can be provided between the elastic elements 30, 40, 50, for example, generally in a metatarsal region of the prosthetic foot 1. Advantageously, in some embodiments the fastening member's 60 position can be adjustable along the length of the slit 36. For example, when the fastening member 60 is a bolt and nut, the bolt can be moved to any desired position along the slit 36 and then fastened into place by tightening the nut. In some embodiments, an undercut or recess in the elastic members 30, 40, 50 can be provided to prevent the bolt and nut from protruding outwards. Notably, the position of the fastening member along the slit 36 can alter the flexibility and resistance of the elastic members 30, 40, 50. Where the elastic members 30, 40, 50 are not held together (e.g., by the fastening member) they can separate and act as distinct elastic members instead of combining into a single elastic member where held together. Thus, if the fastening member 60 is moved forward, the clastic members 30, 40, 50 are held together over a shorter range, allowing more separation between them, and thus greater flexibility (e.g., the lever arm of the second elastic member 40 is relatively longer, resulting in greater flexibility of the prosthetic foot 1). Alternatively, if the fastening member is moved rearward, the elastic members 30, 40, 50 are held together over a longer range, reducing the allowed separation and flexibility (e.g., the lever arm of the second elastic member 40 is relatively shorter, resulting in increased stiffness of the prosthetic foot 1). Advantageously, the fastening member 60 can be adjusted to vary the stiffness of the prosthetic foot 1.

In some embodiments, the stiffness control member 60 can be mechanically actuated, cither manually by the user or automatically (e.g., actively adjusted) during ambulation by the user (e.g., based on the activity level of the user or the phase of gait cycle).

Notably, as discussed above, in some embodiments, the flexibility and resistance of the elastic members 30, 40, 50 can also be altered by the actuator 20 (independently of, or in combination with, the stiffness control member 60). Thus, it will be understood that the flexibility and resistance of the clastic members 30, 40, 50 can be altered manually and/or by an actuator. In further examples, the stiffness control member 60 can be moved (e.g., automatically moved) by an actuator to adjust the resistance and flexibility of the clastic members 30, 40, 50.

In some embodiments, it may be preferable to adjust the flexibility and resistance of the clastic members 30, 40, 50 to reduce resistance and increase flexibility while moving on level ground. Thus, for example, the stiffness control member 60 can be moved forward while ambulating on level ground to provide faster plantarflexion after heel strike. During other gait patterns, such as walking downstairs, one can reduce flexibility and increase resistance by moving the stiffness control member 60 backward. In some embodiments, these gait patterns can be detected by sensors and processors provided on or in communication with the prosthetic foot 1. An actuator can then be controlled to adjust the flexibility and resistance of the elastic members 30, 40, 50 according to the detected gait pattern.

Variations to the embodiment in FIGS. 1-8 are possible. For example, in the depicted embodiment a stiffness control member 60 (e.g., fastening member 60) can be moved to various positions along the slit 36, such that the resistance and flexibility of the elastic members 30, 40, 50 can be varied. However, in other embodiments it may be preferable to remove the slit 36 such that the elastic members 30, 40, 50 are more solid and provide a more uniform resistance. Further, in some embodiments it may be preferable to bond the elastic elements 30, 40, 50 in another manner, such as with an adhesive, so they remain permanently attached. The elastic elements 30, 40, 50 can also be held together with additional fastening members 62, depicted as nuts and bolts in FIG. 8, in addition to the adjustable fastening member 60. In other embodiments, one or more of the elastic elements 30, 40, 50 can be formed together into a single piece. For example, in some embodiments the second and third clastic members 40, 50 can be formed as a single piece.

In further embodiments this resistance can be varied by other methods. For example, in some embodiments the stiffness control member can be a wedge or insert that can be inserted where two or more of the elastic members 30, 40, 50 meet. For example, a wedge can be inserted between the first and second elastic members 30, 40 (e.g., above the second and below the first). Similarly, a wedge can be inserted between the second and third elastic members 40, 50, such as at a wedging location 64, depicted in FIG. 4. The wedge can limit the range of motion of the elastic members 30, 40, 50 relative to each other, thus increasing their resistance. The size and shape of the wedge can be chosen to cause a particular desired resistance. Further, the wedge can be moved forward or rearward to vary the flexibility and resistance between the elastic members 30, 40, 50.

The depicted embodiment also combines three separate elastic elements 30, 40, 50 that each provide a separate function. For example, the first elastic element 30 acts as a spring in parallel with the actuator 20. Further, the second elastic element 40 acts as a spring in series with the actuator 20. Both elastic elements 30, 40 can thus be configured to work with or against the actuator 20 at different phases of the gait cycle. Further, the elastic elements 30, 40 can be loaded or unloaded by the actuator 20. Providing one spring in parallel and the other in series allows each spring to have a different effect on the dynamics of the prosthetic foot 1 during movement. For example, during heel strike, the actuator 20 and second elastic member 40 can act in series to provide the prosthetic foot 1 with a certain level of flexibility in addition to the energy stored by the third elastic member (e.g., be relatively less stiff at heel-strike), while during toe-off, the actuator 20 and first elastic element 30 can act in parallel to provide the prosthetic foot with a different level of flexibility (e.g., be relatively more stiff at toc-off). Thus, the independent flexibility and resistance of the elastic elements 30, 40 can be chosen separately to optimize the behavior of the prosthetic foot 1.

Notably, in the depicted embodiment the first and second elastic members 30, 40 both extend toward the toe along the foot portion 34. However, they do not extend toward the heel of the prosthetic foot 1. The third elastic member 50 includes a heel portion 56. The heel portion 56 thus provides flexibility and resistance to the prosthetic foot 1 during heel strike. This response during heel strike can be determined independently of a flexibility and resistance during toe-strike or toe-off during a gait cycle, as the third elastic element 50 is a separate piece from the first and second elastic elements 30, 40. Thus, for example, a system of separate elastic members 30, 40, 50 can include versions of each elastic member with varying flexibilities and resistances. One can then choose each elastic member 30, 40, 50 to provide a desired flexibility and resistance at different times during a gait cycle, depending on the needs of a particular user.

In further embodiments, the actuator 20 can be removed or replaced with a rigid member. For example, in some embodiments the second elastic member 40 can connect directly to the second connection portion 14. In such embodiments, the first and second elastic members 30, 40 can both be rotatably connected to the attachment member 10. Further, in embodiments where the second elastic member 40 does not connect directly to the second connection portion 14, it can still rotatably connect to an intermediary member (such as a rigid member replacing the actuator 20) at a fourth connection portion 22 (as described above). In such embodiments, the three rotatable connections 14, 16, 22 can form a triangle with at least one elastic portion, the elastic portion being both the first and second elastic members 30, 40, between the fourth connection portion 22 and the third connection portion 16.

The rotatable connections 14, 16, 22 with the elastic members 30, 40 can provide a flexible resistance to rotation of the attachment member 10. Advantageously, the use of both first and second elastic members 30, 40 can provide for a natural rocking motion during stance that can provide improved stability with the prosthetic foot 1. This stability can also be provided in embodiments that include an actuator 20, e.g., when the actuator 20 is locked in a particular position or is substantially inactive.

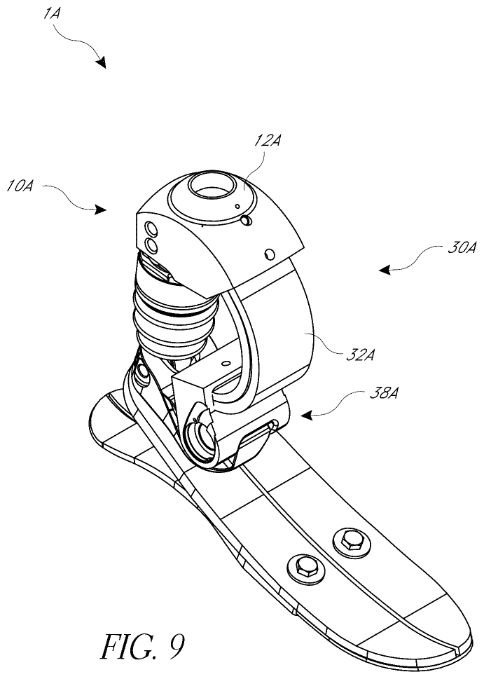

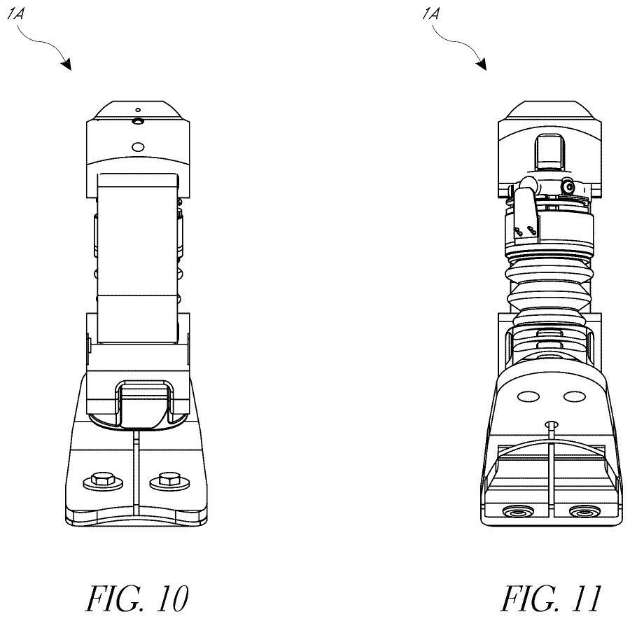

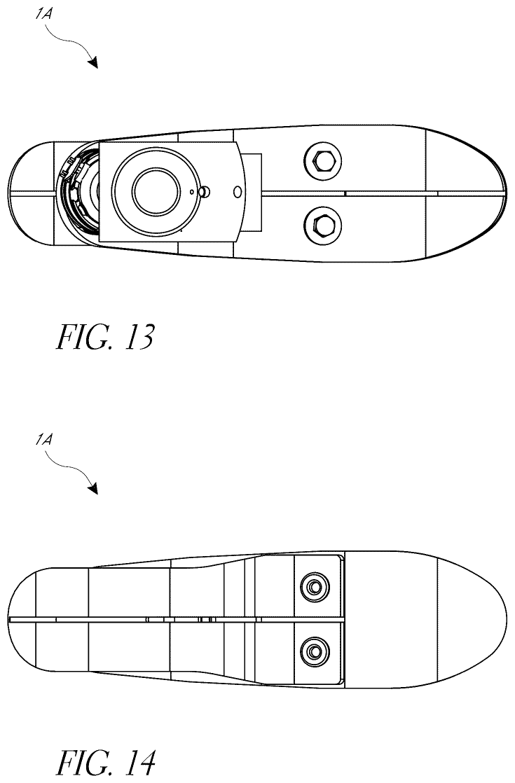

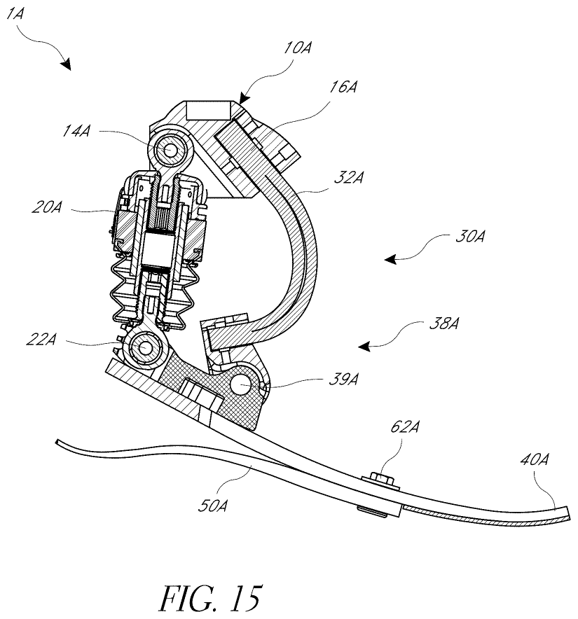

FIGS. 9-15 depict a second embodiment of a prosthetic foot 1A. It will be understood that the prosthetic foot 1A in these figures has features similar to the prosthetic foot 1 described above, and thus will be described in terms of its differences. As shown, the prosthetic foot 1A includes a first elastic member 30A having a C-shaped portion 32A, similar to that in the previously described embodiments. However, in the present embodiment the C-shape portion 32A can be reversed to have an opening facing rearward (e.g., the C-shaped portion 32A has a concave shape facing toward the rear of the prosthetic foot 1). Further, as shown, the first elastic member 30A can include two parallel elastic pieces. Additionally, as shown, the first elastic member 30A can attach to the attachment member 10A at a second connection portion 14A that is non-rotatable, although in other embodiments the second connection portion 14A can be rotatable (e.g., via a pivot location, as shown in previous embodiments). Further, as shown, the first elastic member 30A can be shortened to not include a foot portion, like the foot portion 34 in the previous embodiments. Instead, the first elastic member 30A can attach to a fifth connection body 38A, also via a non-rotatable connection. However, the fifth connection body 38A can provide a rotatable connection to the second clastic member 40A, as shown in the figures, via a supplemental connection body 39A that can be considered to be part of the fifth connection body 38A.

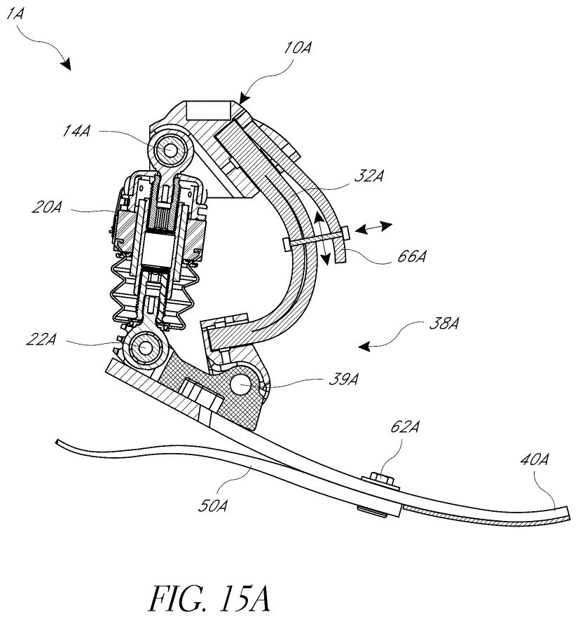

Notably, the features in the embodiment in FIGS. 9-15 also form a triangle with at least one elastic portion and three rotatable connections, similar to that discussed above in the previous embodiment. For example, as best shown in FIG. 15, the prosthetic foot 1A includes a second connection portion 14A between the attachment member 10A and the actuator 20A. The actuator 20A can include a fourth connection portion 22A, connecting to the second elastic member 40A. The second elastic member 40A can connect to the first elastic member 30A with fifth connection body 59A. The first elastic member 30A can attach to the attachment member 10A, completing the triangle. In some embodiments, only one portion of the triangle can have an elastic portion. For example, in some embodiments the second elastic member 40A can be an inelastic or rigid member.

Further, in some embodiments the C-shaped portion 32A can be substantially similar to that shown in FIGS. 9-15, but include an additional elastic member 66A generally aligned with the C-shaped portion 32A, as best shown in FIG. 15A. The additional elastic member 66A can connect to the attachment member 10A in a manner similar to the first elastic member 30A. The additional elastic member 66A can then extend tangent with the first elastic member 30A along the C-shaped portion 32A and terminate at a free end unattached to the fifth connection body 39A. A stiffness control member similar to the stiffness control member 60 can then be provided between the C-shaped portion 32A and the additional clastic member 66A. For example, slits can be provided in the C-shaped portion 32A and the additional elastic member 66A to receive a fastening member that can be moved along the length of the slit to adjust the flexibility and resistance of the C-shaped portion 32A, as illustrated. In further embodiments, such adjustability of the flexibility and resistance of the clastic members 30A, 40A, 50A can be provided with other suitable mechanisms. For example, in some embodiments the fifth connection body 38A (including the supplemental connection body 39A) can be movable in an anterior-posterior direction along the second elastic member 40A to change the location of the fifth connection body 38A on the second elastic member 40A. For example, in some embodiments the second elastic member 40A can include a slot that can receive a fastener to fasten the fifth connection body 38A in place along the second elastic member. However, other suitable mechanisms can be used to adjust the location of the fifth connection body 38A relative to the second elastic member 40A (e.g., a track and worm gear arrangement).

Additionally, the depicted prosthetic foot 1A depicts an alternative method for attaching the second and third elastic members 40A, 50A. As shown, these members can be attached by two bolts 62A, on opposite sides of the slit 36A. However, it will be understood that other attachment methods can be used, such as those described above. Further, an adjustable fastening member can be provided in the slit 36A, as discussed above, to vary the flexibility and resistance of the prosthetic foot 1A.

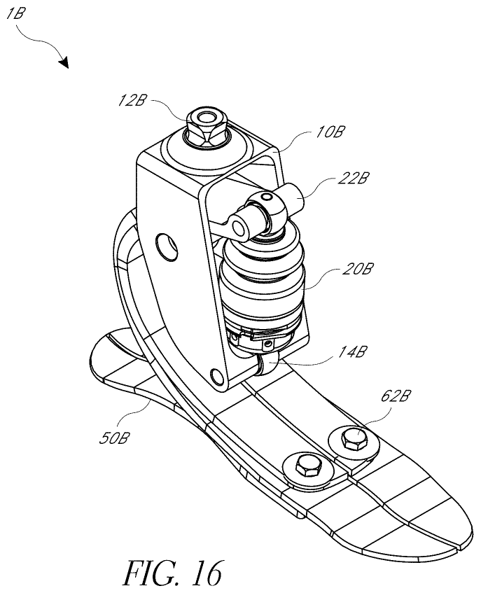

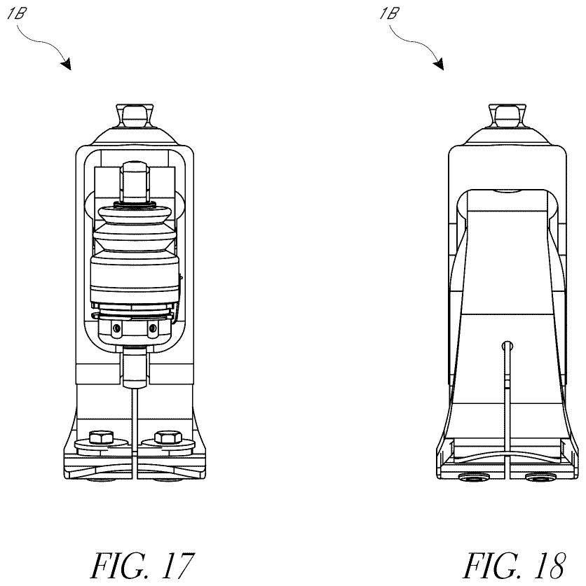

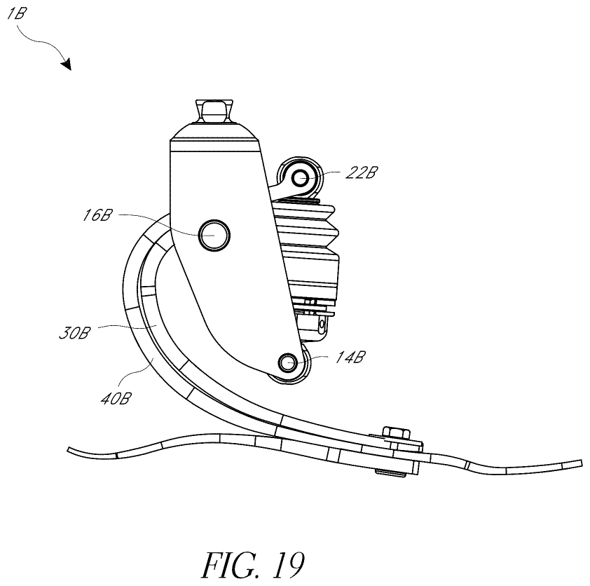

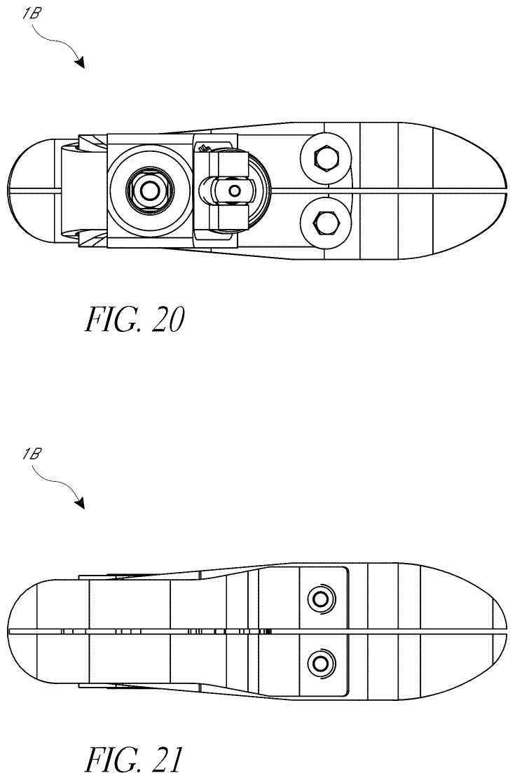

FIGS. 16-23 depict yet another embodiment of a prosthetic foot 1B. It will again be understood that the prosthetic foot 1B in these figures has features similar to the prosthetic foot 1 described above, and thus will be described in terms of its differences. As shown, the prosthetic foot 1B provides a design with the actuator 20B in a forward portion of the prosthetic foot. As shown, the attachment member 10B can still attach to the first elastic element 30B at a rotatable third connection portion 16B. However, the third connection portion 16B can be provided at an upper rear portion of the attachment member 10B. The second connection portion 14B can connect to the actuator 20B at a lower forward portion of the attachment portion 10B. The actuator 20B can then extend upwards from the second connection portion 14B to attach to the second elastic member 40B. The second elastic member 40B can then form a C-shaped portion that follows the forward-facing C-shaped portion 32B in the first elastic member 30B.

Connection of the third elastic member 50B is depicted as being substantially similar to the third elastic member 50A depicted in FIGS. 9-15. Similar variations can also be provided, as discussed above. Further, as shown, the first elastic member 30B can extend to a foot portion 34A and attach to the second and third elastic members 40B, 50B by the same bolts 62B. Further, as shown, the second elastic member 40B can extend beyond the first and second elastic members 30B, 50B, to a toe portion of the prosthetic foot 1B.

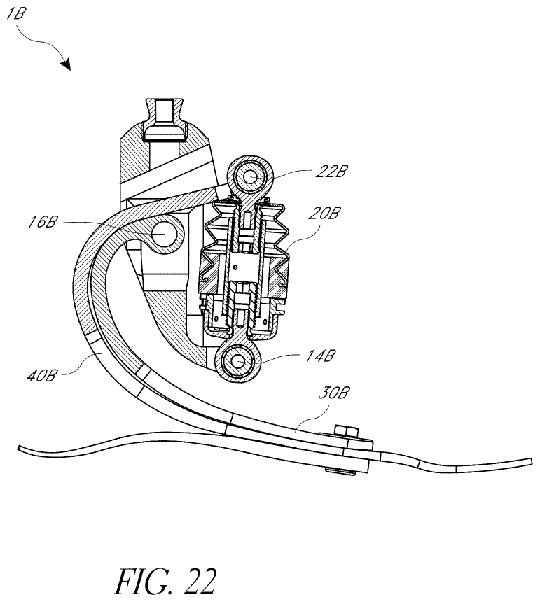

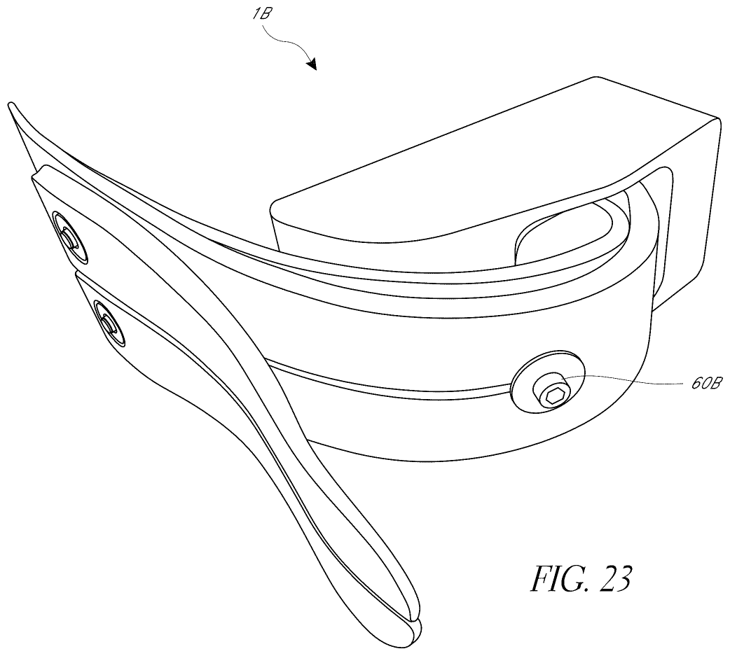

Notably, the features in the embodiment in FIGS. 16-23 also form a triangle with at least one elastic portion and three rotatable connections, similar to that discussed above in the previous embodiments. For example, as best shown in FIG. 22, the prosthetic foot 1B includes a second connection portion 14B between the attachment member 10B and the actuator 20B. The actuator 20B can include a fourth connection portion 22B, connecting to the second elastic member 40B. The second elastic member 40A can come to abut the first elastic member 30B, as described above regarding the previous embodiments. The first elastic member 30B can attach to the attachment member 10B at the third connection portion 16B, completing the triangle.

Further, as best shown in FIG. 23, the first and second elastic members 30B, 40B can be connected by a stiffness control member 60B. In the illustrated embodiment, the stiffness control member 60B is an adjustable fastening member 60B and can allow for varied resistance and flexibility in a manner similar to that in the embodiments discussed above. In the present embodiment, the adjustable fastening member 60B is provided between only the first and second clastic members 30B, 40B (and not the third elastic member 50B), and in a rear portion of the prosthetic foot 1B. Moving the adjustable fastening member 60B downward and forward results in a lever arm of the first and second elastic members 30B, 40B between the fastening member 60B and the attachment member 10B that is relatively longer, resulting in increased flexibility of the prosthetic foot 1B. Alternatively, moving the adjustable fastening member 60B upward results in a lever arm of the first and second elastic members 30B, 40B between the fastening member 60B and the attachment member 10B that is relatively shorter, resulting in increased stiffness of the prosthetic foot 1B.

Advantageously, the prosthetic foot 1, 1A, 1B embodiments disclosed can provide for a natural rocking motion during a stance phase of gait that can provide improved stability to the prosthetic foot 1, 1A, 1B (e.g., the attachment member 10, 10A, 10B can move relative to one or more of the elastic members during stance). This improved stability can also be provided in embodiments that include an actuator 20, 20A, 20B, e.g., when the actuator 20, 20A, 20B is locked in a particular position or is substantially inactive. Additionally, in some embodiments, the prosthetic foot can include a stiffness control member that can be mechanically actuated (e.g., manually or automatically) to vary a stiffness of one or more elastic members of the prosthetic foot to provided different levels of stiffness (e.g., during different types of gait).

Conditional language used herein, such as, among others, “can,” “could,” “might,” “may,” “e.g.,” and from the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment.

While the above detailed description has shown, described, and pointed out novel features as applied to various embodiments, it may be understood that various omissions, substitutions, and changes in the form and details of the ground contact sensing system, including the sensor components, logical blocks, modules, and processes illustrated may be made without departing from the spirit of the disclosure. As may be recognized, certain embodiments of the systems described herein may be embodied within a form that does not provide all of the features and benefits set forth herein, as some features may be used or practiced separately from others. Additionally, features described in connection with one embodiment can be incorporated into another of the disclosed embodiments, even if not expressly discussed herein, and the prosthetic device having the combination of features still fall within the scope of the invention.

1. (canceled)

2. A prosthetic foot comprising:
an attachment member disposed at an ankle position of the prosthetic foot, the attachment member comprising a first connection portion configured to connect the attachment member to a user or another prosthetic device of the user, the first connection portion including a pyramid connector;
an actuator coupled to a posterior portion of the attachment member by a second connection portion at a first end of the actuator;
a first elongate member coupled to a second end of the actuator by a third connection at a heel end of the first elongate member, the third connection being a rotatable connection, the second end of the actuator being below the first end when the prosthetic foot is at rest on a flat surface, wherein the first elongate member extends forward from the heel end of the first elongate member toward a toe end of the prosthetic foot;
a second elongate member disposed below the first elongate member and configured to contact a ground surface when the prosthetic foot is in use, the second elongate member extending from the toe end to a heel end of the prosthetic foot, wherein the second elongate member is fixedly coupled to the first elongate member at a location posterior to the toe end of the prosthetic foot, and wherein the first and second elongate members are operably coupled to an anterior portion of the attachment member by a fourth connection portion; and
one or more sensors configured to measure characteristics of the prosthetic foot or an environment of the prosthetic foot; and
a processing system configured to receive data from the one or more sensors and communicate with an external device to transfer data from the one or more sensors to the external device.

3. The prosthetic foot of claim 2, wherein the one or more sensors comprise a position or movement sensor, a pressure sensor, a rotation sensor, and/or a ground contacting sensor.

4. The prosthetic foot of claim 2, wherein the processing system is in wired communication with the external device.

5. The prosthetic foot of claim 2, wherein the processing system is in wireless communication with the external device.

6. The prosthetic foot of claim 2, wherein the environment measured by the one or more sensors comprises a terrain on which the prosthetic foot moves.

7. The prosthetic foot of claim 2, wherein the characteristics of the prosthetic foot measured by the one or more sensors comprise a gait parameter.

8. The prosthetic foot of claim 2, wherein the characteristics of the prosthetic foot measured by the one or more sensors comprise an activity level of the user of the prosthetic foot.

9. The prosthetic foot of claim 2, wherein the processing system is located within the attachment member.

10. The prosthetic foot of claim 9, wherein the attachment member comprises a housing cover.

11. The prosthetic foot of claim 2, wherein the attachment member provides a rigid connection between the first connection portion, the second connection portion, and the fourth connection portion.

12. A prosthetic foot comprising:
an attachment member disposed at an ankle position of the prosthetic foot, the attachment member comprising a first connection portion configured to connect the attachment member to a user or another prosthetic device of the user, the first connection portion including a pyramid connector;
an actuator coupled to a posterior portion of the attachment member by a second connection portion at a first end of the actuator, the actuator being active or powered;
a first elongate member coupled to a second end of the actuator by a third connection at a heel end of the first elongate member, the third connection being a rotatable connection, the second end of the actuator being below the first end when the prosthetic foot is at rest on a flat surface, wherein the first elongate member extends forward from the heel end of the first elongate member toward a toe end of the prosthetic foot;
a second elongate member disposed below the first elongate member and configured to contact a ground surface when the prosthetic foot is in use, the second elongate member extending from the toe end to a heel end of the prosthetic foot, wherein the second elongate member is fixedly coupled to the first elongate member at a location posterior to the toe end of the prosthetic foot, and wherein the first and second elongate members are operably coupled to an anterior portion of the attachment member by a fourth connection portion; and
one or more sensors configured to measure characteristics of the prosthetic foot or an environment of the prosthetic foot,
wherein the actuator is configured to be controlled by a processing system on or in communication with the prosthetic foot to adjust flexibility or resistance of the first elongate member and the second elongate member based on the measured characteristics of the prosthetic foot or the environment of the prosthetic foot.

13. The prosthetic foot of claim 12, further comprising communication electronics located on the attachment member, the communication electronics configured to receive data from the one or more sensors and communicate with an external device to transfer the data from the one or more sensors to the external device.

14. The prosthetic foot of claim 13, wherein the attachment member comprises a housing cover.

15. The prosthetic foot of claim 13, wherein the communication electronics are wired communication with the external device.

16. The prosthetic foot of claim 13, wherein the communication electronics are in wireless communication with the external device.

17. The prosthetic foot of claim 12, wherein the one or more sensors comprise a position or movement sensor, a pressure sensor, a rotation sensor, and/or a ground contacting sensor.

18. The prosthetic foot of claim 12, wherein the environment measured by the one or more sensors comprises a terrain on which the prosthetic foot moves.

19. The prosthetic foot of claim 12, wherein the characteristics of the prosthetic foot measured by the one or more sensors comprise a gait parameter.

20. The prosthetic foot of claim 12, wherein the characteristics of the prosthetic foot measured by the one or more sensors comprise an activity level of the user of the prosthetic foot.

21. The prosthetic foot of claim 12, wherein the attachment member provides a rigid connection between the first connection portion, the second connection portion, and the fourth connection portion.
